(19) United States
(12) Patent Application Publication
(10) Pub. No.: US 2016/0120729 A1
(43) Pub. Date: May 5, 2016

(54) DEEP VEIN THROMBOSIS PROHPYLAXIS

(71) Applicant: Regents of the University of Minnesota, Minneapolis, MN (US)

(72) Inventors: James A. Ankrum, Iowa City, IA (US); James D. Cezo, Colorado Springs, CO (US)

(21) Appl. No.: 14/931,457

(22) Filed: Nov. 3, 2015

Related U.S. Application Data
(60) Provisional application No. 62/074,392, filed on Nov. 3, 2014.

(51) Int. Cl. A61H 1/02; A61B 17/132; A61H 9/00

(52) U.S. Cl. CPC A61H 1/0237; A61H 9/0007; A61B 17/1322; A61B 17/1327; A61H 2209/00; A61H 2205/10

(57) ABSTRACT

A compression device to be applied to an appendage, including a generally non-stretch sleeve sized to fit the appendage having a first edge and a second edge and a plurality of stretch portions operably coupled to either the first edge or the second edge. A plurality of substantially non stretch tensioning members is operably coupled to one of the plurality of stretch portions and releasably adjustably coupleable to the generally non-stretch sleeve so as to apply tension to the stretch portions and to the generally non-stretch sleeve whereby compression is applied to the appendage.

### RELATED APPLICATION

The present application claims the benefit of U.S. Provisional Application No. 62/074,392, filed Nov. 3, 2014 and entitled “Deep Vein Thrombosis Prophylaxis”, the entire contents of which, including the appendix, is incorporated by reference herein.

### FIELD OF THE INVENTION

The invention relates to both static and dynamic mechanical compression as a prophylaxis measure for the prevention of deep vein thrombosis.

### BACKGROUND OF THE INVENTION

Deep vein thrombosis (DVT) is the third leading cause of cardiovascular death in the United States and effects approximately 600,000 patients each year. Patients are at high risk of DVT after surgery or trauma and require DVT prophylactic measures. DVT prophylaxis can be divided into three main categories, pharmaceutical prophylaxis, static mechanical compression and dynamic mechanical compression. While devices currently on the market are effective at preventing DVT, they are dependent on patient compliance and have critical drawbacks that reduce their consistent use by patients and healthcare providers. Particularly, current devices are difficult to put on, hot, itchy, and require custom fitting to each patient. In addition, most dynamic mechanical compression devices are also bulky, limiting patient's range of motion and interfering with clothing. Most dynamic mechanical compression devices also tether the patient to a machine due to their reliance on pneumatic hoses.

Current static compression device technologies rely heavily on circumferential elastic stockings. As the level of stretch of the stocking material determines the level of compression on the limb, frequent fittings are required to deliver the prescribed compression level and gradient. These devices are also prone to create reverse gradients (compression higher in the proximal portion of the limb than the distal portion) due to errors in fitting. As they are very tight, compression stockings are difficult to put on and can be very itchy and uncomfortable.

Current dynamic compression systems rely heavily on inflatable chambers to create compressive zones. These systems are bulky and must be tethered to an air pump to function.

Accordingly, there is still room for improvement the area of deep vein thrombosis prophylaxis.

### SUMMARY OF THE INVENTION

Embodiments of the invention overcome many of the above identified barriers to patient compliance. A device according to an example embodiment of the invention is configured to apply both static and dynamic compression. The devices of the invention have a slim form factor allowing them to be easily worn under clothes. An example device is initially loose and elastic making it easy to put on. The device is made of moisture and heat wicking fabrics to reduce patient discomfort. The level of compression can be adjusted by the patient, in one example embodiment of the invention, with the twist of a dial just below the knee. Embodiments of the invention are initially loose fitting, allowing the patient to easily place the device. Embodiments of the invention may be fit to the patient using the twist.

In the dynamic configuration, according to an example embodiment, pulses of higher compression are delivered by a small actuator or motor supplied by onboard battery power that limits patient tethering.

According to an example embodiment the device includes a calf sleeve having a tubular structure with two segments. The two segments can be formed of two separate fabrics, one fabric resistant to stretch and one fabric that is easily stretchable joined together. Alternately, the invention can employ a single fabric construction in which a first region of the fabric has a weave that allows stretch while a second region is resistant to stretch. When used in relation to the lower leg, the non-stretch fabric wraps around the back of the leg while the stretchy segment lies against the anterior lateral portion of the shin.

A dial based cable tensioner, for example like that available from Boa Technologies, is mounted on the non-stretch fabric, for example, at the top of the sleeve just below the knee. A cable from the tensioner extends out across the stretchy segment to lace guides mounted to the non-stretchy segment. The cable criss-crosses the stretchy segment from lace guide to lace guide mounted on the non-stretchy segment.

At the distal end of the sleeve, the cable enters a small housing where, in a static embodiment, the cable is attached to a constant force spring. The spring anchors the distal end of the cable and limits the maximum tension in the cable.

In a dynamic embodiment, this distal housing contains an actuator which captures the cable and is configured to increase or decrease the tension in the cable to create dynamic waves of compression applied to the calf. The housing also contains a mechanical gauge that provides the user either qualitative or quantitative feedback to the level of compression. An electronic or other gauge can be used as well.

The lace guides are designed and aligned on the device to create a continuously variable compression gradient along the length of the calf. By modulating the number of lace guides, the length of the lace guide, and the number of passes of the cable between two paired lace guides, a level of compression in a discrete segment can be tuned. The lace guides are formed from low friction materials and the cable exit from the guide is elevated from the surface of the sleeve to prevent the cable from rubbing against the patient's leg. Embodiments of the invention are initially loose, allowing the patient to easily place the device. The compression device is fit to the patient using the twist dial and can be adjusted at anytime by the patient without requiring intervention by a healthcare professional.

Embodiments of the current invention are also designed to be comfortable with moisture and heat wicking materials and a form factor that allows the device to be worn under normal clothing. The dynamic embodiment includes on board power for an actuator that provides variation in compression and does not require tethering to an outside power source or pump.

According to another embodiment, the deep vein thrombosis prophylactic device which can be referred to as the DVTP device includes a combination of stretching and non-stretching fabric. While all fabric stretches to some degree, in accordance with the invention, non-stretch fabric shall be considered to be fabrics that are woven and designed to stretch minimally under tension applicable by manual manipulation by the human hand while stretchy fabrics show noticeable stretching at levels of tension that can be readily achieved by manual manipulation by patients.

Accordingly, embodiments of the invention generally include a non-stretch sleeve portion, a stretching portion and a webbing or strap portion.

Generally, according to embodiments of the invention, webbing, straps or cables are pulled to tighten the DVT device and the webbing straps or cables are secured by a tensioner, a snap or buckle.

According to one embodiment of the invention, the no stretch sleeve portion includes a partial circumference generally cylindrical or tapered cylindrical shape. This portion of the invention is sized to fit over the limb of the patient to be treated prophylactically. Generally this would be the calf of the leg but other appendages may be treated as well.

According to an embodiment of the invention, the stretch portion includes multiple portions, approximately half of which are secured to one edge of the non-stretch sleeve portion and another half are secured to an opposing edge of the non-stretch sleeve portion. A relatively non-stretch strap, webbing or cable extends from a first edge through an aperture in the stretch portion and then extends back to the first edge. On a portion of the sleeve to which the stretch portion is secured to the first edge, the strap extends from the second edge through an aperture in the stretch portion and then back to the second edge where a free end of the strap is secured by a snap, buckle, hook and loop fasteners or other fastener means known to those skilled in the art. According to an embodiment of the invention, the stretch portions are equal in number and alternately secured to either the first edge or the second edge.

According to an alternate configuration, a strap, webbing or cable portion is secured to the stretch portion which is, in turn, secured to either the first or second edge of the no stretch sleeve portion. The cable, strap or webbing includes a buckle or clip which can be moved along the length of the cable, strap or webbing to change the effective length of the cable, strap or webbing and thus the degree of tension applied by stretching the stretch portion.

According to another embodiment of the invention, the no stretch sleeve portion is structured to overlap and thus completely encircle the appendage to be treated. This eliminates gaps present in a previously discussed embodiment.

According to an embodiment of the invention, a series of pass-through slits may be formed in the no stretch sleeve portion. The pass-through slits are sized so that the webbing strap or cable may pass through them while being wrapped around the no stretch sleeve portion. The no stretch sleeve portion may have a generally tapered structure when viewed unfurled and laid flat. A clip or buckle can be secured on the strap, webbing or cable so that a first portion of the strap, webbing or cable secured to a first end of the no stretch sleeve portion may be secured to a second portion of the strap, webbing or cable secured to a second end of the no stretch sleeve portion. Accordingly, in this embodiment, the no stretch sleeve portion encircles the appendage to be treated with some overlap while the first and second strap portions encircle the appendage a second time and are secured to each other on a side of the appendage generally opposing the location of the pass-through slits.

According to another embodiment of the invention, the strap, cable or webbing portions may be secured to a stretch portion which, is in turn, secured to a first or second edge of the no stretch sleeve portion of the DVT device. According to another embodiment of the invention, pass-through slits may be staggered alternately or in another pattern along the no stretch sleeve portion of the DVT device.

According to another embodiment of the invention, at least some of the straps, webbing or cables can be interconnected by a bridge member.

In accordance with embodiments of the invention, a generally non-stretch fabric is considered to be a fabric which exhibit less than 80% strain at 5 lbs of force on a 2 inch wide piece of the fabric. Stretchable fabrics, which often contain elastic fibers, can stretch over 100% strain under these same conditions and purely non-stretch materials stretch less than 20% at these conditions. Another variation of fabric used in this invention is short stretch fabric. Short stretch fabric, for the purposes of this application is considered to be a fabric which exhibits a low force in response to strains up to 50%, but for which strains beyond 50% forces increase substantially.

The application of the sleeve onto an extremity includes the following steps. First, the sleeve is loosely fitted over the extremity with straps or cabling fully elongated. Once the sleeve is in place over the extremity, the straps or cabling can be tightened to remove slack from the fabric of the device. Next, the straps are tightened as needed to provide a circumferential pressure on the extremity. The straps are tightened such that a pressure gradient is created from distal to proximal. Finally, the straps can be loosened to enable easy removal of the device.

The sizing of the sleeve, according to example embodiments, is such that it can accommodate a large range of the population and therefore, fewer sizes of the device are expected to be needed to serve the patient population. Devices according to the prior art are sized such that they provide therapeutic compression for an extremity within a circumference range of 2-5 inches. For instance, according to the prior art, a medium compression stocking for 20-30 mmHg compression may only be useable for legs which have a calf circumference in the 14-19 inch range. In accordance with example embodiments of the invention, the sizing of the sleeve enables compression therapy for extremities with more than 5 inches of difference in circumference. In an example embodiment, the sleeve is useable on a calf which has a range of 13-21 inches in circumference. To accommodate that range, an embodiment of the device as portrayed in FIG. 3 is dimensioned such that the width of the fabric at the proximal end is 24 inches, the width of the fabric at the distal end is 15 inches, and the straps are up to 18 inches long.

The invention may have application in conditions including, but not limited to, deep vein thrombosis prophylaxis, postthrombotic syndrome symptom relief, athletic massage, venous insufficiency and dependent edema.

The invention is expected to enhance patient compliance by making it compatible with everyday life. Embodiments of the invention are simple, easy to put on, easy to adjust and easy to fit. The device is comfortable, does not require a tether and can be worn in any environment.

### BRIEF DESCRIPTION OF THE DRAWINGS

FIG. 1 is a perspective view of a deep vein thrombosis prophylaxis device in a rolled configuration according to an embodiment of the invention;

FIG. 2 is a perspective view of a deep vein thrombosis device according to another embodiment of the invention;

FIG. 3 is a plan view of the device of FIG. 2 in an unrolled configuration;

FIG. 4 is a plan view of a deep vein thrombosis device in an unrolled configuration according to another embodiment of the invention;

FIG. 5A is a schematic view of a stretch indicator in a relatively unstretched configuration according to an embodiment of the invention;

FIG. 5B is a schematic view of the stretch indicator of FIG. 5A in a more stretched configuration;

FIG. 6 is a schematic plan view of an alternate webbing configuration according to an embodiment of the invention; and

FIG. 7 is a schematic top view of a deep vein thrombosis prophylaxis device according to an embodiment of the invention depicting the relative orientation of slits, buckles and straps.

### DETAILED DESCRIPTION OF THE DRAWINGS

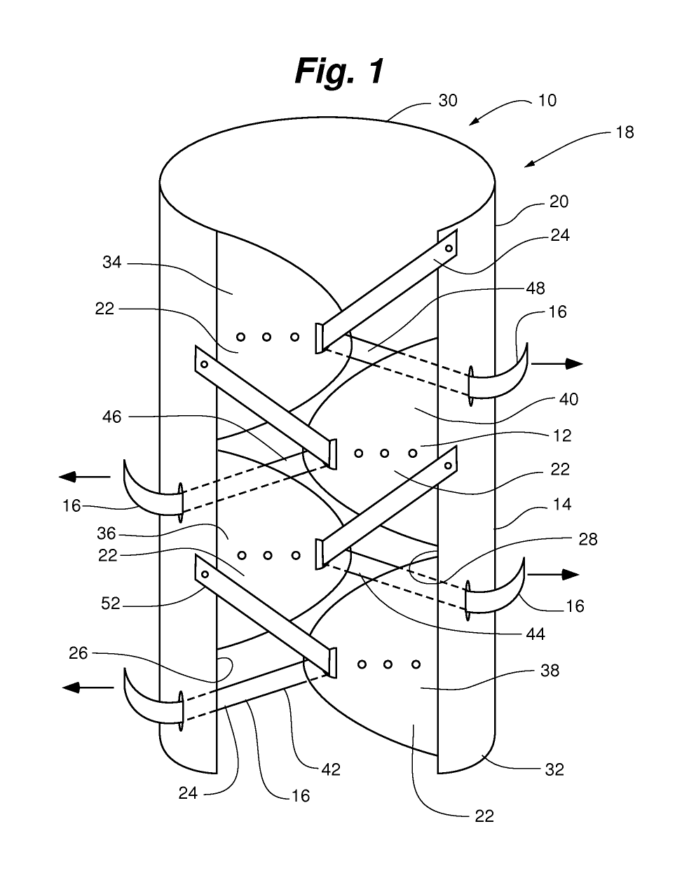

Referring particularly to FIGS. 1-7, DVT prophylaxis device 10 generally includes portions of stretch material 12, portions of non-stretch material 14 and tensioning members 16.

Referring particularly to FIG. 1, deep vein thrombosis device (DVTD) 18 generally includes non-stretch sleeve portion 20, stretch portion 22 and tensioning straps 24.

Non-stretch sleeve portion 20, as depicted in FIG. 1, generally includes a partially tubular structure bounded by first edge 26, second edge 28, upper border 30 and lower border 32. In the embodiment depicted in FIG. 1, first stretch member 34 and second stretch member 36 are secured to first edge 26. Third stretch member 38 and fourth stretch member 40 are secured to second edge 28. DVTD 18 further includes first tension strap 42, second tension strap 44, third tension strap 46 and fourth tension strap 48. In the depicted embodiment, first tension strap 42 extends from first edge 26, passes through strap aperture 50 and returns to first edge 26. A first end 52 of first tension strap 42 is fixedly secured to second edge 28. Second end 54 of first tension strap 42 generally includes connector element 56. Connector element 56 may be a snap, buckle or snap and loop connector, for example.

Second tension strap 44, third tension strap 46 and fourth tension strap 48 are structured similarly to first tension strap 42.

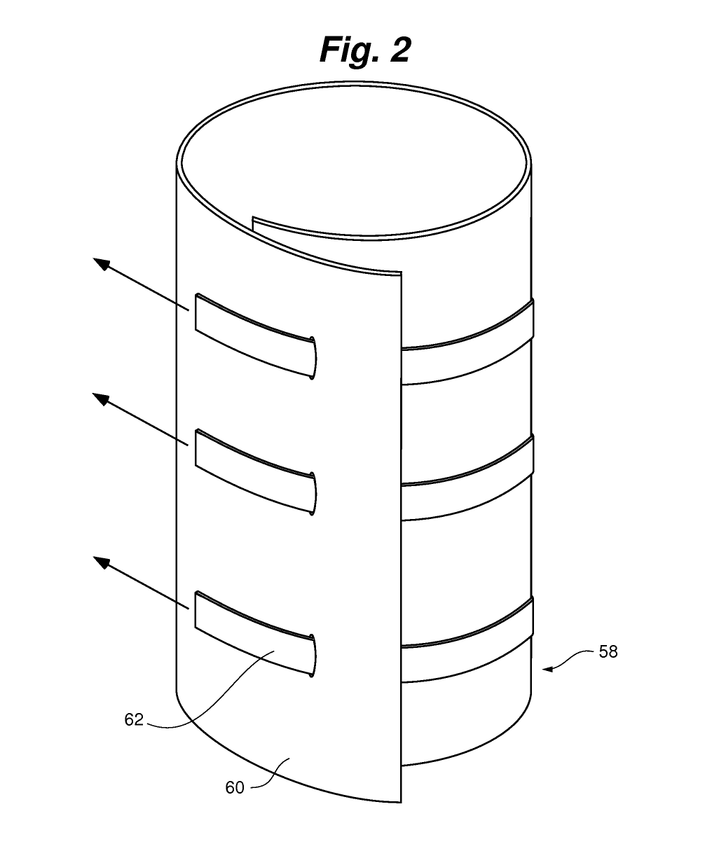

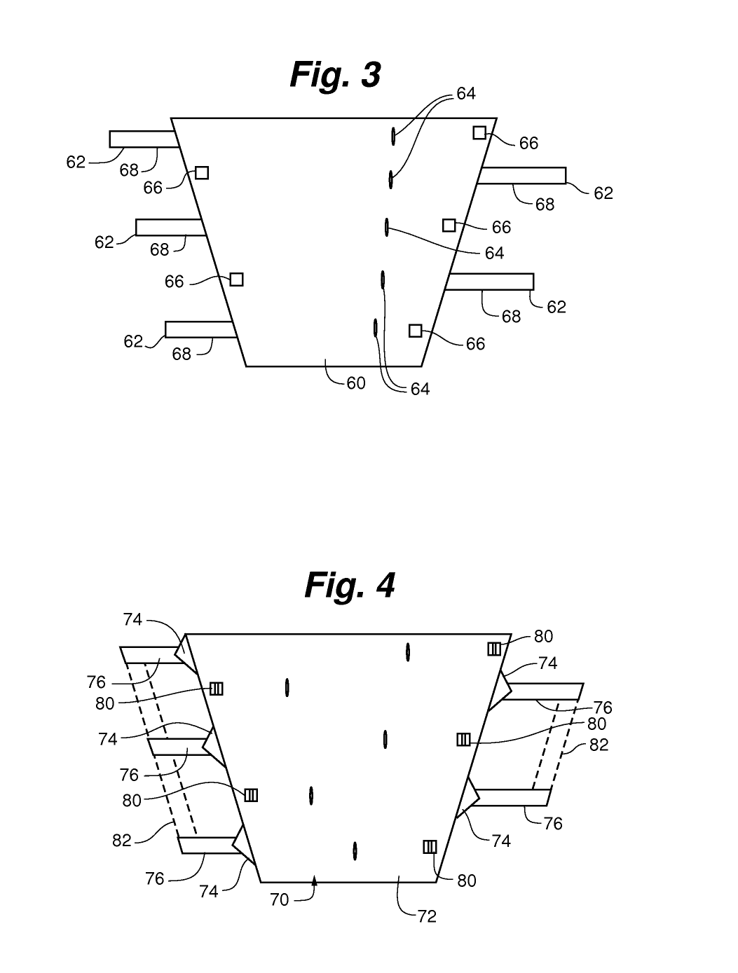

Referring now to FIGS. 2 and 3, another embodiment of the invention is depicted. DVTD 58 generally includes non-stretch sleeve portion 60 and tensioning straps 62.

Referring particularly to FIG. 3, non-stretch sleeve portion 60 has a generally trapezoidal tapered shape. Non-stretch sleeve portion 60 is pierced by pass-through slits 64 and supports connector elements 66. According to another embodiment of the invention, tensioning straps 62, as depicted in FIGS. 2 and 3, may be formed of a stretchable strap material 68. Tensioning straps 62 are generally aligned with pass-through slits 64 which, in turn, are generally aligned with connector elements 66.

Referring now to FIG. 4, another embodiment of DVTD 70 is depicted. The depicted embodiment includes non-stretch sleeve portion 72, stretch portions 74 and tensioning straps 76. Similar to the earlier discussed embodiment, non-stretch sleeve portion 72 has a generally tapered trapezoidal shape and is pierced by pass-through slits 78. Unlike the earlier depicted embodiment, pass-through slits 78 here are staggered in an alternating pattern. In the depicted embodiment, non-stretch sleeve portion also supports connector elements 80.

As depicted in dashed lines, according to another embodiment of the invention, bridge element 82 couples some of tensioning straps 76. Bridge element 82 thus facilitates ease of tightening and adjustment by allowing adjustment of multiple tensioning straps 76 at a time.

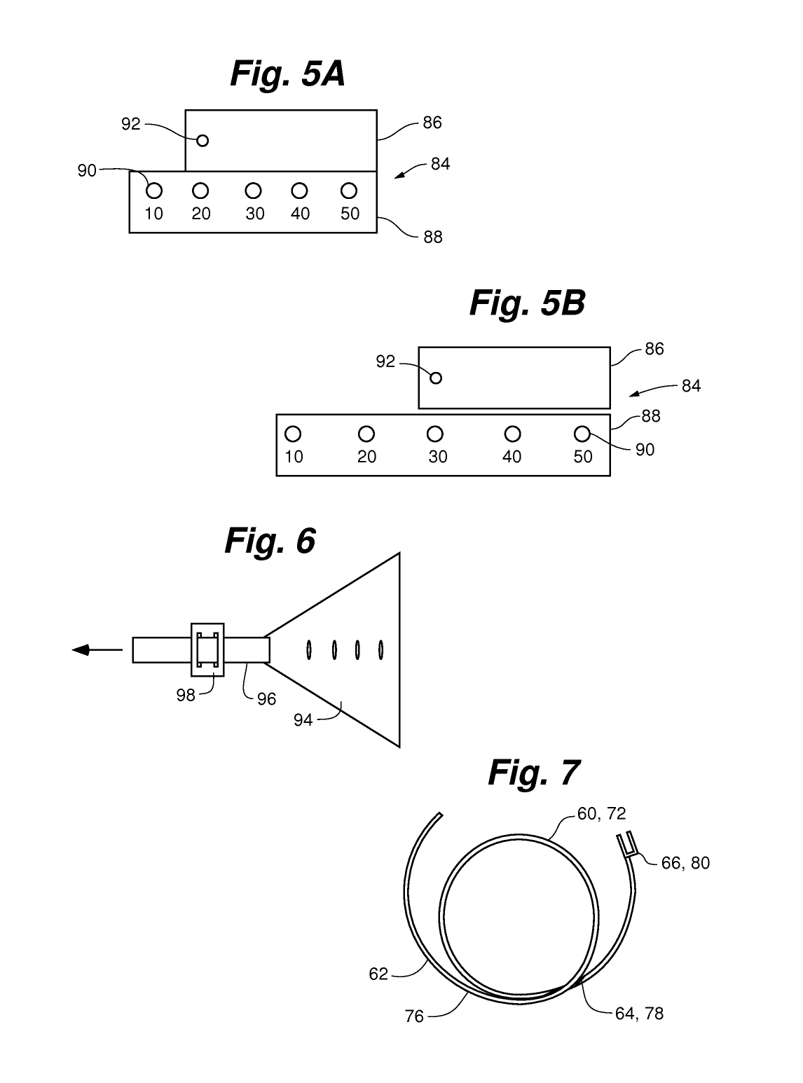

Referring particularly to FIGS. 5A and 5B, any embodiment of the invention may include stretch indicator 84. Stretch indicator 84 generally includes fixed portion 86 and stretchable portion 88. Stretchable portion 88 supports numerical indices 90 while fixed portion 86 supports measuring index 92. As tension is applied to stretchable portion 88, numerical indices 90 are spread apart in a proportional fashion so that reading numerical index 90 that is adjacent measuring index 92 a level of stretch and thus a level of tension can be identified.

Referring to FIG. 6, an alternative embodiment of stretch portion 94 and tensioning element 96 is depicted. In the depicted embodiment, tensioning strap 96 includes movable connector element 98 thereon. Moveable connector element 98 is slidably adjustable or otherwise adjustable along the length of tensioning strap 96 thus allowing adjustment and tensioning of tensioning strap 96.

Referring now to FIG. 7, a top sectional schematic view of the relationship of tensioning straps 62 and 76, pass-through slits 64, and 78, connector elements 66 and 80 and non-stretch sleeve portions 60 and 72 is depicted. Tensioning straps 62 and 76 are passed through pass-through slits 64 and 78 passed around the exterior of non-stretch sleeve portions 60 and 72 and coupled together via connector elements 66 and 80.

The present invention may be embodied in other specific forms without departing from the spirit of the essential attributes thereof; therefore, the illustrated embodiments should be considered in all respects as illustrative and not restrictive, reference being made to the appended claims rather than to the foregoing description to indicate the scope of the invention.

1. A compression device to be applied to an appendage, comprising:
a non-stretch sleeve sized and shaped to fit the appendage and to at least partially encircle the appendage, the non-stretch sleeve having a first edge and an opposing second edge;
a plurality of stretch portions, each of the plurality of stretch portions being operably coupled to either the first edge or the second edge;
a plurality of non-stretch tensioning members, including a first tensioning member and a second tensioning member, each of the plurality of non-stretch tensioning members being operably coupled at a first end portion thereof to one of the plurality of stretch portions and releasably, adjustably coupleable at a second end portion thereof to the non-stretch sleeve so as to apply tension to the stretch portions and to the non-stretch sleeve whereby compression is selectively applied to the appendage.

2. The compression device as claimed in claim 1, wherein at least one of the plurality of non-stretch tensioning members passes through an opening in the stretch portion and returns to the first edge or the second edge from which the tensioning member originates.

3. The compression device as claimed in claim 1, wherein at least one of the plurality of non-stretch tensioning members member passes through an opening in the non-stretch sleeve.

4. The compression device as claimed in claim 1, wherein at least one of the plurality of non-stretch tensioning members further comprises a connector element that releasably operably couples the at least one non-stretch tensioning member to the non-stretch sleeve.

5. The compression device as claimed in claim 3, wherein at least one of the plurality of non-stretch tensioning members is sized and constructed to encircle the non-stretch sleeve when the non-stretch sleeve is applied to the appendage and to releasably attach to another non-stretch tensioning member.

6. The compression device as claimed in claim 1, further comprising a stretch indicator.

7. The compression device as claimed in claim 6, further wherein the stretch indicator comprises a fixed portion and a stretchable portion, a numerical index and a measuring index wherein the numerical index is coupled to one of the fixed portion and the stretchable portion and the measuring index is coupled to the other of the fixed portion and the stretchable portion.

8. The compression device as claimed in claim 1, wherein variable tension of the tensioning members is achieved by tension member spacing that establishes a tension gradient of the sleeve whereby a pressure gradient is achieved wherein pressure is greater distally than proximally on the appendage and whereby blood is urged to return toward a heart.

9. A method of applying therapeutic compression to an appendage, comprising:
applying to the appendage a non-stretch sleeve sized to fit the appendage, the non-stretch sleeve having a first edge and an opposing second edge;
applying tension to the non-stretch sleeve via a plurality of stretch portions operably coupled to either the first edge or the second edge;
applying tension to the plurality of stretch portions by tensioning a plurality of non-stretch tensioning members operably coupled to the stretch portions at a first portion thereof, including a first tensioning member and a second tensioning member;
securing a second portion of at least one of the non-stretch tensioning members to the non-stretch sleeve; and
thereby applying therapeutic compression to the appendage.

10. The method as claimed in claim 10, further comprising passing at least one of the plurality of tensioning members through an opening in the stretch portion and coupling the second portion of the tensioning member o the first edge or the second edge from which the tensioning member originates.

11. The method as claimed in claim 10, further comprising passing at least one of the plurality of tensioning members through an opening in the non-stretch sleeve.

12. The method as claimed in claim 10, further comprising coupling a connector element of the at least one of the plurality of tensioning members to the non-stretch sleeve.

13. The method as claimed in claim 10, further comprising:
wrapping at least one of the plurality of tensioning members to encircle the non-stretch sleeve when the non-stretch sleeve is applied to the appendage; and
releasably attaching the at least one of the plurality of tensioning members to another the plurality of tensioning members.

14. The method as claimed in claim 10, further comprising adjusting tension of at least one of plurality of tensioning members by consulting a numerical index and a measuring index of a stretch indicator.

15. The method as claimed in claim 10, further comprising establishing a tension gradient of the sleeve whereby a pressure gradient is achieved wherein pressure is greater distally than proximally on the appendage by tension member spacing.

16. A compression device to be applied to an appendage, comprising:
a sleeve sized to fit the appendage, the sleeve having a first edge and an opposing second edge
the sleeve being a unitary structure having a non-stretch sleeve portion and at least one stretch sleeve portion;
a plurality of non-stretch tensioning members, including a first tensioning member and a second tensioning member, each of which is operably coupled at a first end portion thereof to the stretch portion and at a second end portion thereof to the non-stretch portion so as to apply tension to the stretch portions and to the non-stretch portions whereby compression is selectively applied to the appendage;
wherein the non-stretch tensioning members are releasably, adjustably coupleable at one or both of the first end portion or the second end portion.

17. The compression device as claimed in claim 16, wherein the first tensioning member is sized and constructed to encircle the sleeve when the sleeve is applied to the appendage and to releasably attach to the second tensioning member.

18. The compression device as claimed in claim 16, further comprising a stretch indicator.

19. The compression device as claimed in claim 16, wherein variable tension of the tensioning members is achieved by tension member spacing that establishes a tension gradient of the sleeve whereby a pressure gradient is achieved wherein pressure is greater distally than proximally on the appendage and whereby blood is urged to return toward a heart.

20. The compression device as claimed in claim 16, wherein the tensioning member passes through an opening in the sleeve.

21. A compression device to be applied to an appendage, comprising:
a non-stretch sleeve sized and shaped to fit the appendage and to at least partially encircle the appendage, the non-stretch sleeve having a first edge and an opposing second edge;
a plurality of non-stretch tensioning members, each of the plurality of non-stretch tensioning members being operably coupled to either the first edge or the second edge;
the plurality of non-stretch tensioning members, including a first tensioning member and a second tensioning member, each of the plurality of non-stretch tensioning members being operably coupled at a first end portion thereof to a first portion of the non-stretch sleeve and releasably, adjustably coupleable at a second end portion thereof to a second portion of the non-stretch sleeve so as to apply tension to the non-stretch sleeve whereby compression is selectively applied to the appendage such that a compression gradient exists wherein the compression is greater at a distal portion of the appendage and lesser at a proximal portion of the appendage.

22. The compression device as claimed in claim 21, the non-stretch sleeve further comprising a plurality of stretch portions and each of the plurality of non-stretch tensioning members being operably coupled at a first end portion thereof to one of the plurality of stretch portions and releasably, adjustably coupleable at a second end portion thereof to the non-stretch sleeve so as to apply tension to the stretch portions and to the non-stretch sleeve whereby compression is selectively applied to the appendage

23. The compression device as claimed in claim 22, wherein at least one of the plurality of non-stretch tensioning members passes through an opening in the stretch portion and returns to the first edge or the second edge from which the tensioning member originates.

24. The compression device as claimed in claim 21, wherein at least one of the plurality of non-stretch tensioning members member passes through an opening in the non-stretch sleeve.

25. The compression device as claimed in claim 21, wherein at least one of the plurality of non-stretch tensioning members further comprises a connector element that releasably operably couples the at least one non-stretch tensioning member to the non-stretch sleeve.

26. The compression device as claimed in claim 24, wherein at least one of the plurality of non-stretch tensioning members is sized and constructed to encircle the non-stretch sleeve when the non-stretch sleeve is applied to the appendage and to releasably attach to another non-stretch tensioning member.

27. The compression device as claimed in claim 21, further comprising a stretch indicator.

28. The compression device as claimed in claim 27, further wherein the stretch indicator comprises a fixed portion and a stretchable portion, a numerical index and a measuring index wherein the numerical index is coupled to one of the fixed portion and the stretchable portion and the measuring index is coupled to the other of the fixed portion and the stretchable portion.

29. The compression device as claimed in claim 1, wherein variable tension of the tensioning members is achieved by tension member spacing that establishes a tension gradient of the sleeve whereby a pressure gradient is achieved wherein pressure is greater distally than proximally on the appendage and whereby blood is urged to return toward a heart.
